(19) United States
(12) Patent Application Publication
(10) Pub. No.: US 2022/0417401 A1
(43) Pub. Date: Dec. 29, 2022

(54) ENDOSCOPE AND IMAGING MODULE

(71) Applicant: OLYMPUS CORPORATION, Tokyo (JP)

(72) Inventors: Keiichi KOBAYASHI, Sagamihara-shi (JP); Takatoshi IGARASHI, Minowa-machi (JP)

(73) Assignee: OLYMPUS CORPORATION, Tokyo (JP)

(21) Appl. No.: 17/897,539

(22) Filed: Aug. 29, 2022

Related U.S. Application Data
(63) Continuation of application No. PCT/JP2020/008855, filed on Mar. 3, 2020.

(51) Int. Cl. H04N 5/225; G02B 23/24; H01L 27/146

(52) U.S. Cl. CPC H04N 5/22521; G02B 23/243; H01L 27/14634; H04N 2005/2255; A61B 1/04

(57) ABSTRACT

An endoscope comprising: an objective optical system; an imager having a light receiving plane that faces an emitting plane of the objective optical system; a semiconductor device provided so as to face a plane, of the imager, opposite to the light receiving plane; and a conductive member that covers the objective optical system, the imager, and the semiconductor device, the conductive member having an external dimension that is identical between a side of the objective optical system and a side of the imager. A distance from an end portion of the semiconductor device in a direction orthogonal to an optical axis of the objective optical system to an inner wall of the conductive member is shorter than a distance from an end portion of the imager in the direction orthogonal to the optical axis of the objective optical system to the inner wall of the conductive member.

### CROSS REFERENCE TO RELATED APPLICATION

This application is a continuation of International Patent Application No. PCT/JP2020/008855, having an international filing date of Mar. 3, 2020, which designated the United States, the entirety of which is incorporated herein by reference.

### BACKGROUND

As a measure to cope with static electricity in an imaging module used in an endoscope, there is known a method using a conductor to release static electricity to a ground. For example, Japanese Unexamined Patent Application Publication No. 2019-25207 discloses an endoscope including a tip flange portion, a lens unit, an image sensor, and a linear conductor. The tip flange portion is attached on an objective side of the lens unit, the image sensor is provided on the opposite side, the tip flange portion is connected to one end of the linear conductor, and the linear conductor is connected from the tip flange portion via the outside of the lens unit to an earth portion.

### SUMMARY

In accordance with one of some aspect, there is provided a An endoscope comprising:

an objective optical system;

an imager having a light receiving plane that faces an emitting plane of the objective optical system;

a semiconductor device provided so as to face a plane, of the imager, opposite to the light receiving plane; and

a conductive member that covers the objective optical system, the imager, and the semiconductor device, the conductive member having an external dimension that is identical between a side of the objective optical system and a side of the imager, wherein

a distance from an end portion of the semiconductor device in a direction orthogonal to an optical axis of the objective optical system to an inner wall of the conductive member is shorter than a distance from an end portion of the imager in the direction orthogonal to the optical axis of the objective optical system to the inner wall of the conductive member.

In accordance with one of some aspect, there is provided an imaging module comprising:

an objective optical system;

an imager having a light receiving plane that faces an emitting plane of the objective optical system;

a semiconductor device provided so as to face a plane, of the imager, opposite to the light receiving plane; and

a conductive member that covers the objective optical system, the imager, and the semiconductor device, the conductive member having an external dimension that is identical between a side of the objective optical system and a side of the imager, wherein

a distance from an end portion of the semiconductor device in a direction orthogonal to an optical axis of the objective optical system to an inner wall of the conductive member is shorter than a distance from an end portion of the imager in the direction orthogonal to the optical axis of the objective optical system to the inner wall of the conductive member.

### BRIEF DESCRIPTION OF THE DRAWINGS

FIG. 1 is a cross-sectional view illustrating a configuration of an imaging module according to a first embodiment.

FIG. 2 is a cross-sectional view of a conductive member and a semiconductor device.

FIG. 3 is a cross-sectional view of a conductive member and a semiconductor device.

FIG. 4 illustrates an example of a ground line pattern provided on the semiconductor device.

FIG. 5 illustrates an example of an electrostatic protection device provided in the semiconductor device.

FIG. 6 is a cross-sectional view illustrating a configuration of an imaging module according to a second embodiment.

FIG. 7 is a cross-sectional view illustrating a configuration of an imaging module according to a third embodiment.

FIG. 8 illustrates a bottom plate and a semiconductor device viewed in a Z direction.

FIG. 9 illustrates a bottom plate and a semiconductor device viewed in the Z direction.

FIG. 10 is a cross-sectional view illustrating a configuration of an imaging module according to a fourth embodiment.

FIG. 11 illustrates an imager, the semiconductor device, and the conductive member view in a −Z direction.

FIG. 12 is a cross-sectional view illustrating a configuration of an imaging module according to a sixth embodiment.

FIG. 13 illustrates a detailed configuration example of a sensor section and a laminated body.

FIG. 14 is a circuit block diagram of the imager and the laminated body.

FIG. 15 illustrates a configuration example of an endoscope system including an endoscope incorporating the imaging module.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. First Embodiment

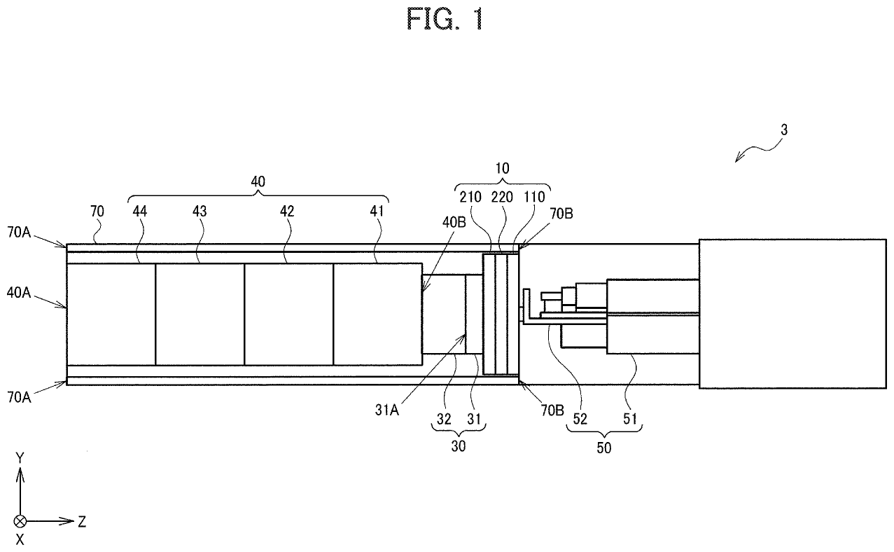

FIG. 1 is a cross-sectional view illustrating a configuration of an imaging module 3 according to a first embodiment. In FIG. 1, an optical axis direction of the imaging module 3 is represented as a Z direction. An X direction and a Y direction are respectively orthogonal to the Z direction and orthogonal to each other. Note that directions opposite to the X, Y, and Z directions are referred to as −X, −Y, and −Z directions, respectively. That is, FIG. 1 is a cross-sectional view of the imaging module 3 taken along a plane parallel to a YZ plane.

The imaging module 3 includes an objective optical system 40, a sensor section 30, a laminated body 10, a conductive member 70, and a cable section 50. The sensor section 30 includes a cover glass 32 and an imager 31. Note that although FIG. 1 illustrates the imaging module 3 including the laminated body 10, it is sufficient that the imaging module 3 includes one or more semiconductor devices in addition to the imager 31. Furthermore, the number of semiconductor device included in the laminated body 10 is not limited to three, and the number may be two or more.

Please note in the following description that drawings based on embodiments are schematic, and a relation between a thickness and a depth of each section, a ratio of the thicknesses of each section, a relative angle, and the like are different from those in the actual imaging module 3. Also, between the drawings, a relation of each dimension or ratio may be different in some portions. Furthermore, illustration of some components is omitted in some drawings.

The imager 31 has a light receiving plane 31A that faces an emitting plane 40B of the objective optical system 40. Among semiconductor devices 110, 210, and 220 constituting the laminated body 10, the semiconductor device 110 disposed in a rearmost plane is provided so as to face a place opposite to the light receiving plane 31A of the imager 31. The conductive member 70 covers the objective optical system 40, the imager 31, and the laminated body 10. Furthermore, a distance from an end portion of the laminated body 10 to an inner wall of the conductive member 70 is shorter than a distance from an end portion of the imager 31 to the inner wall of the conductive member 70.

More specifically, external diameters of the semiconductor devices 110, 210, and 220 are larger than an external diameter of the imager 31. The external diameter of the semiconductor device is a distance from a center of the semiconductor device to an end portion, of the semiconductor device, furthest to the center of the semiconductor device. When the center of the semiconductor device corresponds to a center of the imager 31, the external diameter of the semiconductor device is a distance from the center of the imager 31 to an end portion, of the semiconductor device, farthest to the center of the imager 31. Furthermore, it also can be said that the external diameter of the semiconductor device is a radius or a diameter of a circle circumscribing the semiconductor device in an XY plane. The external diameter of the imager 31 is a distance from the center of the imager 31 to an end portion, of the imager 31, farthest to the center of the imager 31. Furthermore, it also can be said that the external diameter of the imager 31 is a radius or a diameter of a circle circumscribing the imager 31 in the XY plane.

The external diameters of the semiconductor devices 110, 210, and 220 being larger than the external diameter of the imager 31 can satisfy a condition that the distance from the end portion of the laminated body 10 to the inner wall of the conductive member 70 is shorter than the distance from the end portion of the imager 31 to the inner wall of the conductive member 70.

Note that it is sufficient that any one of the semiconductor devices 110, 210, and 220 included in the laminated body 10 has the distance from the end portion of the semiconductor device to the inner wall of the conductive member 70 being shorter than the distance from the end portion of the imager 31 to the inner wall of the conductive member 70. That is, it is sufficient that at least one of the semiconductor devices 110, 210, and 220 has an external shape larger than an external shape of the imager 31. In the first embodiment, the semiconductor devices 110, 210, and 220 viewed in the Z direction are identical in shapes and sizes, and distances from the respective end portions of the semiconductor devices 110, 210, and 220 to the inner wall of the conductive member 70 are identical.

In Japanese Unexamined Patent Application Publication No. 2019-25207 described above, a linear conductor is provided as a measure to cope with static electricity applied to a scope tip portion, but the image sensor itself is not directly protected by the linear conductor. Accordingly, static electricity protection from a direction in which the linear conductor is not formed is not ensured. Therefore, there is a problem of a need to further ensure protection of the image sensor from static electricity.

According to the present embodiment, the static electricity that has entered the imaging module 3 moves along the conductive member 70 and flows to the laminated body 10 closer to the inner wall of the conductive member 70 than the imager 31. As described later, the laminated body 10 is connected to a ground line of the cable section 50, and the static electricity is grounded from the laminated body 10 to the ground line. As a result, the static electricity that has entered the imaging module 3 is not applied to the imager 31, and it becomes possible to prevent breakage of the imager 31 due to the static electricity.

Furthermore, according to the present embodiment, covering the imager 31 with the conductive member 70 enables protection of the imager 31 not only from the static electricity entering from a tip of the imaging module 3 but also from the static electricity entering from a lateral side of the imaging module 3. For example, there is a risk in use of an endoscope that static electricity that has entered the endoscope enters a lateral side of the imaging module 3 via a device other than the imaging module 3 provided at an endoscope tip. According to the present embodiment, it is possible to protect the imager 31 from such static electricity. Alternatively, there is a risk in manufacturing, specifically, a process of assembling the imaging module 3 to the endoscope tip or the like, that the static electricity enters from the lateral side of the imaging module 3. By adopting a procedure of preparing the imaging module 3 to which the conductive member 70 has been attached, for example, and then assembling the imaging module 3 to the endoscope tip, it is possible to protect the imager 31 from the static electricity generated in manufacturing.

Furthermore, the imaging module can be downsized compared with protection using the linear conductor as in Japanese Unexamined Patent Application Publication No. 2019-25207. That is, in Japanese Unexamined Patent Application Publication No. 2019-25207, the linear conductor is disposed outside the lens unit, and the imaging module is increased in size by the liner conductor. According to the present embodiment, the conductive member 70 itself covering the objective optical system 40 has a static electricity protection function, and therefore the imaging module can be downsized compared with a case of providing the linear conductor.

Furthermore, according to the present embodiment, the objective optical system 40, the imager 31, and the laminated body 10 are entirely covered with the conductive member 70, and therefore a stress input to the imaging module 3 is distributed by the conductive member 70. As a result, loads to connection portions between members are distributed and a risk of breaking the imaging module 3 by the stress is decreased, which leads to improvement in reliability of the imaging module 3.

Furthermore, according to the present embodiment, the imager 31 and the laminated body 10 are covered with the conductive member 70, and therefore a noise input to the imaging module 3 is electrostatically shielded by the conductive member 70, which leads to improvement of an S/N ratio of an image signal. For example, in use of the endoscope, when a device, such as a high-frequency knife, becoming a noise source is used, the conductive member 70 decreases an effect given by the noise to the image signal.

Hereinafter, details of the first embodiment will be described. The objective optical system 40 is an optical system that forms an image of a subject in the light receiving plane 31A of the imager 31. The objective optical system 40 includes a plurality of lenses 41 to 44 arranged side by side in the Z direction. The objective optical system 40 is a laminated lens in which a plurality of lenses made of, for example, glass or plastic is laminated. Of two planes at both ends of the objective optical system 40, a plane where light from the subject enters in use of the endoscope is represented as an entering plane 40A, and a plane where the light is emitted is represented as an emitting plane 40B. The entering plane 40A and the emitting plane 40B are planes parallel to the XY plane, but may be planes curved due to the surface shape of the lens.

The cover glass 32 is a member that protects the light receiving plane 31A of the imager 31, and has a first plane that contacts the emitting plane 40B and a second plane that is provided to an opposite side to the first plane and contacts the light receiving plane 31A. The imager 31 is a sensor that captures a two-dimensional image, and is constituted by a semiconductor chip integrating pixel arrays and control circuits. A circuit configuration of the imager 31 will be described later together with a circuit configuration of the laminated body 10.

The laminated body 10 is a lamination of semiconductor devices as a plurality of layers. FIG. 1 illustrates an example where the semiconductor devices 110, 210, and 220 of three layers are laminated. Each of the semiconductor devices 110, 210, and 220 is a semiconductor chip in which circuits for operating the imager 31 are integrated. Substrate planes of the semiconductor chips are referred to as main planes. A main plane on the objective side is referred to as a first main plane, and a main plane opposite to the first main plane is referred to as a second main plane. A thickness direction of the substrate of the semiconductor chip is parallel to the Z direction, and the first main plane and the second main plane are parallel to the XY plane. The first main plane of the semiconductor device 210 faces and contacts the plane opposite to the light receiving plane 31A of the imager 31. The first main plane of the semiconductor device 220 contacts the second main plane of the semiconductor device 210, and the first main plane of the semiconductor device 110 contacts the second main plane of the semiconductor device 210. Shapes viewed in the Z direction of the semiconductor devices 110, 210, and 220 are, for example, rectangular. The semiconductor devices 110, 210, and 220 of three layers are laminated so that sides of the semiconductor devices conform to each other when viewed in the Z direction.

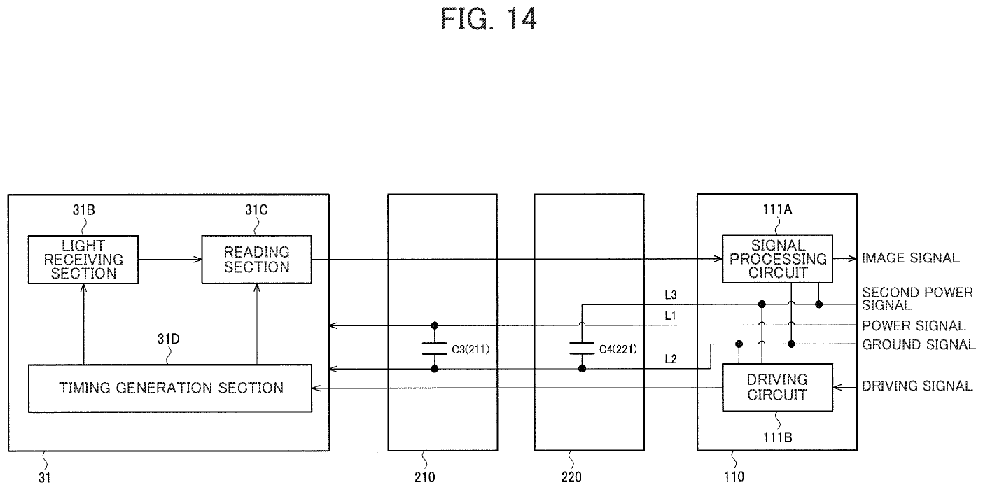

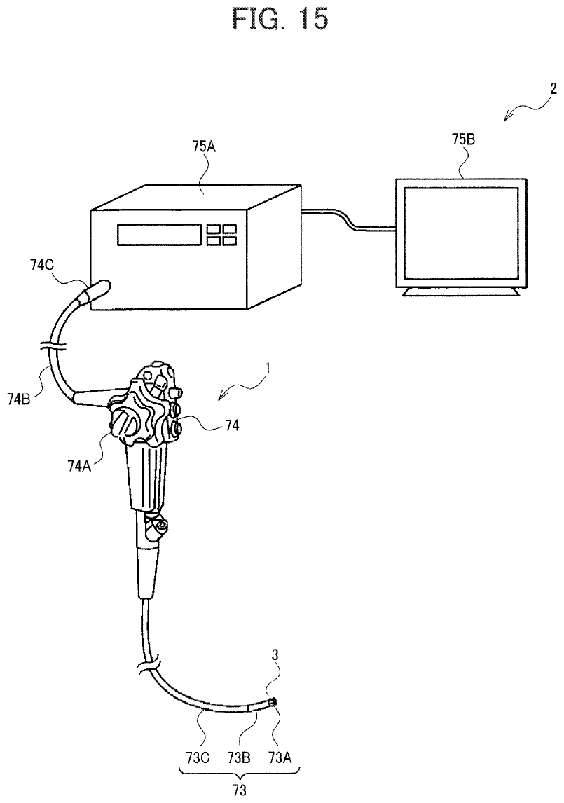

One end of the cable section 50 is connected to the second main plane of the semiconductor device 110. The cable section 50 includes, for example, a signal cable 51 and an FPC substrate 52. The second main plane of the semiconductor device 110 is provided with a plurality of terminals. The plurality of terminals is connected to the signal cable 51 via the FPC substrate 52. For example, in a circuit example described later referring to FIG. 14, a power signal, a second power signal, a ground signal, and a driving signal are input from the cable section 50 to the terminals of the semiconductor devices 110, and those signals are input to the imager 31 via the laminated body 10. Furthermore, the image signal read from the imager 31 is output via the laminated body 10 from the terminals of the semiconductor device 110 to the cable section 50. In a state where the imaging module 3 is assembled in the endoscope, the other end of the cable section 50 is connected to a connector for connecting the endoscope to a processor. For example, in an example of an endoscope system 2 described later referring to FIG. 15, the signal cable 51 is extended via a universal code 74B to a connector 74C.

The conductive member 70 is constituted by a conductive material such as, for example, metal or carbon. Furthermore, the conductive member 70 has a light shielding property. By the light shielding property of the conductive member 70, the objective optical system 40 and the imager 31 covered with the conductive member 70 are shielded from light. As a result, the imaging module 3 can be improved in its optical performance. For example, it is possible to suppress image abnormality such as a flare generated when light enters the objective optical system 40.

The conductive member 70 is shaped like a cylinder extending in the Z direction. The conductive member 70 has, for example, a rectangular or circular shape in a cross section parallel to the XY plane, and its cross section and a thickness of its wall plane are uniform irrespective of their positions in the Z direction. Note that the shape of the cylinder is not limited to this. For example, the thickness of the wall plane of the cylinder may be non-uniform, the wall plane of the cylinder may be provided with one or a plurality of holes, or the wall plane of the cylinder may be structured by a net-like member. Furthermore, the conductive member 70 is not necessarily be entirely conductive. For example, a conductive thin film may be deposited to a side plane of a cylinder made of a non-conductor such as resin, for example.

Of both ends of the cylinder of the conductive member 70, an end portion on the side of the entering plane 40A is represented as an entering-side end portion 70A, and an end portion on the side of the emitting plane 40B is represented as an emitting-side end portion 70B. The entering-side end portion 70A is in the same plane as the entering plane 40A, for example. Note that the position of the entering-side end portion 70A and the position of the entering plane 40A in the Z direction may be different from each other. For example, the entering-side end portion 70A may be positioned on a −Z direction side compared with the entering plane 40A. The emitting-side end portion 70B is in the same plane as the second main plane of the semiconductor device 110, for example. Note that the position of the emitting-side end portion 70B and the position of the emitting plane 40B in the Z direction may be different from each other. For example, the emitting-side end portion 70B may be positioned at an arbitrary position between the first main plane of the semiconductor device 210 and the second main plane of the semiconductor device 110. In other words, it is sufficient that the conductive member 70 covers at least a part of the laminated body 10, and the end portion on the Z direction side of the laminated body 10 is not necessarily covered.

Note that, inside the cylinder of the conductive member 70, a first sealing resin may be provided. That is, a space between the conductive member 70 and the sensor section 30, the objective optical system 40, and the laminated body 10 may be filled with the first sealing resin. Furthermore, around the cable section 50, a second sealing resin may be provided. That is, a space between the cable section 50 and a member covering the cable section 50 may be filled with the second sealing resin. The first sealing resin and the second sealing resin may be the same type of resin or may be different types of resin. For example, the first sealing resin may be a resin whose viscosity is lower than that of the second sealing resin. For being put afterward in the space between the conductive member 70 and the sensor section 30 and the like, the first sealing resin has preferably low viscosity in view of facility of filling.

Hereinafter, description will be made to the “distance” with the semiconductor device 110 as an example. The meaning of the “distance” is the same as in the imager 31 and the semiconductor devices 210 and 220.

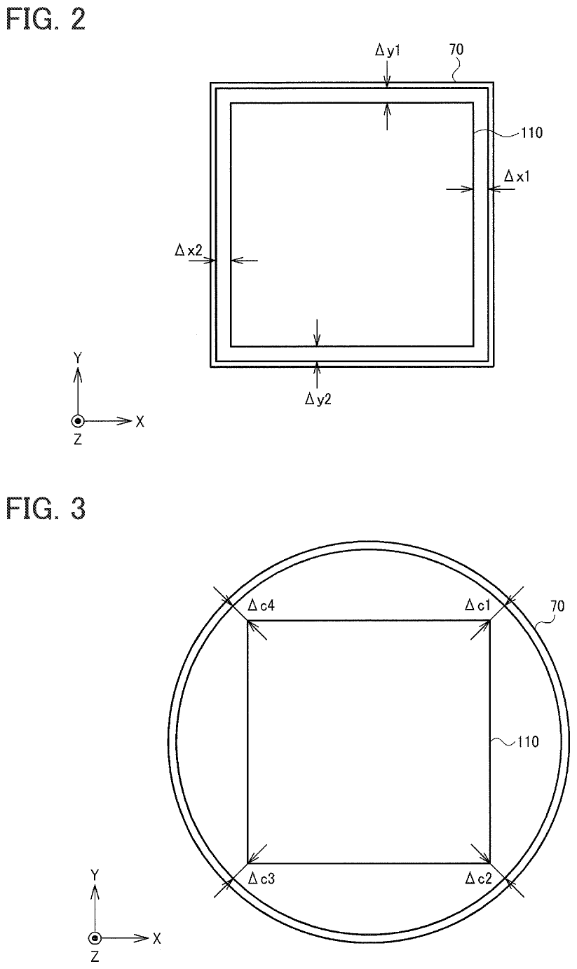

The distance from the end portion of the semiconductor device 110 to the inner wall of the conductive member 70 is the shortest distance between the end portion of the semiconductor device 110 and the inner wall of the conductive member 70. That is, the distance from the end portion of the semiconductor device 110 to the inner wall of the conductive member 70 is the shortest distance among the distances from points of the end portion of the semiconductor device 110 to the inner wall of the conductive member 70. Hereinafter, an example of the shortest distance is shown referring to FIG. 2 and FIG. 3.

FIG. 2 is a cross-sectional view in a case where a cross section of the conductive member 70 and the semiconductor device 110 are square. FIG. 2 illustrates a cross-sectional view of the imaging module 3 taken in a cross section that is parallel to the XY plane and passes through the semiconductor device 110. In FIG. 2, end portions of the semiconductor device 110 correspond to the outer circumference of the semiconductor device 110, i.e., sides of the square. Because there are four sides, distances Δx1, Δx2, Δy1, and Δy2 between the four sides and the inner wall of the conductive member 70 exist. The shortest distance among the four distances is the distance from the end portion of the semiconductor device 110 to the inner wall of the conductive member 70. Note that FIG. 2 illustrates a case where the following relation is satisfied: Δx1=Δx2=Δy1=Δy2.

FIG. 3 illustrates a cross-sectional view in a case where a cross section of the conductive member 70 is circular and the semiconductor device 110 is square. FIG. 3 illustrates a cross-sectional view of the imaging module 3 taken in a cross section that is parallel to the XY plane and passes through the semiconductor device 110. In FIG. 3, end portions of the semiconductor device 110 are corners of the square. Because there are four corners, distances Δc1, Δc2, Δc3, and Δc4 between the four corners and the inner wall of the conductive member 70 exist. The shortest distance among the four distances is the distance from the end portion of the semiconductor device 110 to the inner wall of the conductive member 70. Note that FIG. 3 illustrates a case where the following relation is satisfied: Δc1=Δc2=Δc3=Δc4.

Note that, in the first embodiment, the shortest distance exists within the cross section parallel to the XY plane, whereas the shortest distance in the embodiment described later may be a distance in the Z direction. That is, as long as the distance is the shortest distance between the end portion of the semiconductor device 110 and the inner wall of the conductive member 70, the distance may be within any plane or in any direction. Furthermore, the “direction” includes a case where the distance is zero. That is, the inner wall of the conductive member 70 may be in contact with any one of the semiconductor devices 110, 210, and 220 included in the laminated body 10.

Furthermore, in FIG. 1, in a state of the imaging module 3 as a single body, i.e., in a state before the imaging module 3 is incorporated in the endoscope tip, the conductive member 70 covers the objective optical system 40, the sensor section 30, and the laminated body 10. However, it is sufficient that the state as illustrated in FIG. 1 is implemented at least with the imaging module 3 being incorporated in the endoscope tip. For example, the state of FIG. 1 may be implemented by providing in advance the conductive member 70 in a portion of the endoscope tip where the imaging module 3 is to be incorporated, and inserting the objective optical system 40, the sensor section 30, and the laminated body 10 into the conductive member 70.

### 2. Ground Line Pattern

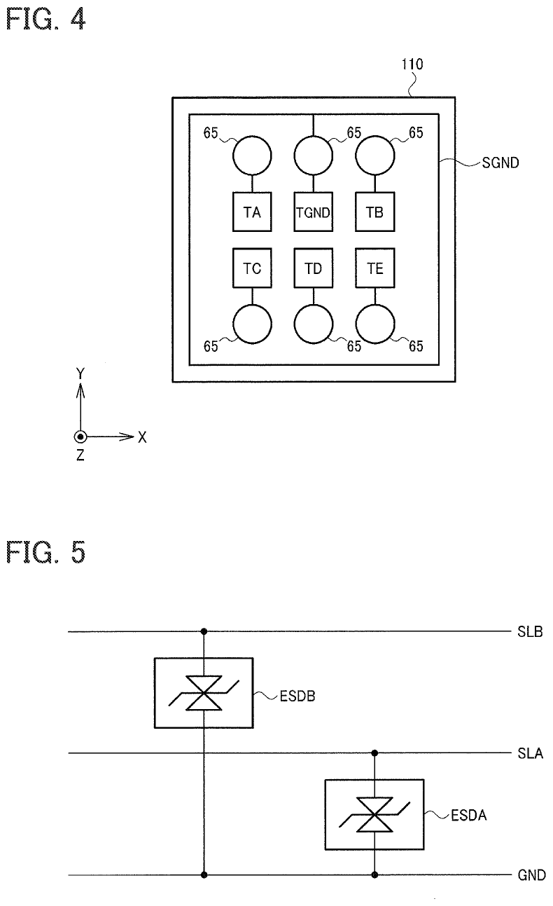

FIG. 4 is an example of a ground line pattern provided on the semiconductor device. FIG. 4 illustrates the second main plane of the semiconductor device 110 viewed in the Z direction. Referring to FIG. 4, description is made to an example where the ground line pattern is provided in the semiconductor device 110. However, the ground line pattern may be provided in the semiconductor devices 210 and 220. Furthermore, the ground line pattern may be provided in all of or some of the semiconductor devices 110, 210, and 220.

The semiconductor device 110 is provided at a marginal edge of the main plane of the semiconductor device 110 and has a ground line SGND connected to ground potential. The semiconductor device 110 has a first main plane that faces a plane opposite to the light receiving plane 31A of the imager 31 and a second main plane that is a plane opposite to the first main plane. The ground line SGND is provided at the marginal edge of at least one of the first main plane and the second main plane. FIG. 4 illustrates an example where the ground line SGND is provided in the second main plane.

Note that the plane being “opposite” to the plane represents the arrangement where the plane faces the plane. More specifically, that the plane being “opposite” to the plane represents that, in a normal line direction of one plane, the other plane exists. The planes may face each other with another element or the like being interposed therebetween. For example, in FIG. 1, the semiconductor devices 210 and 220 are provided between the first main plane of the semiconductor device 110 and the light receiving plane 31A of the imager 31.

According to the present embodiment, the ground line SGND connected to the ground potential is provided at the marginal edge of the main plane of the semiconductor device 110. Accordingly, the distance between the inner wall of the conductive member 70 and the ground line SGND can be made shorter than the distance between the inner wall of the conductive member 70 and another wiring, element, or the like. As a result, it is possible to increase the probability that the static electricity that has entered the conductive member 70 flows from the inner wall of the conductive member 70 to the ground line SGND, and to make the static electricity directly flow to the ground without intermediation of another wiring, element, or the like. Hereinafter, details of FIG. 4 will be described. In the second main plane of the semiconductor device 110, a terminal TGND is provided, and the terminal TGND is connected to the ground line of the cable section 50. Furthermore, in the second main plane of the semiconductor device 110, terminals TA, TB, TC, TD, and TE and a bump 65 are provided. The terminals TA, TB, TC, TD, and TE are connected to a power line or a signal line of the cable section 50. The terminals TGND and TA, TB, TC, TD, and TE are connected to a bump on the side of the first main plane by a through-silicon via.

That is, the marginal edge of the main plane is a region along the outer circumference of the main plane, and includes not only the outer circumference of the main plane but also a neighboring region of the outer circumference. That is, the ground line SGND may be provided either at the outer circumference of the main plane or slightly inside the outer circumference of the main plane. For example, when viewed in the Z direction, the ground line SGND is provided at a position inside the outer circumference of the semiconductor device 110 and outside the outer circumference of the imager 31. In FIG. 4, the ground line SGND circles the marginal edge once without a cutting part. However, the ground line SGND may not necessarily circle the marginal edge completely and may partially include the cutting part.

### 3. Electrostatic Protection Device

FIG. 5 is an example of an electrostatic protection device provided in the semiconductor devices 110, 210, and 220. Note that the electrostatic protection device may be provided to either each of the semiconductor devices 110, 210, and 220, or only some of the semiconductor devices. Hereinafter, description will be made to a case where the electrostatic protection device is provided in the semiconductor device 110.

As illustrated in FIG. 5, the semiconductor device 110 has an electrostatic protection device ESDA. The semiconductor device 110 has a line SLA to which a signal or power is input. The electrostatic protection device ESDA is connected between the line SLA and the ground line GND connected to the ground potential. The semiconductor device 110 may further include one or more electrostatic protection devices. FIG. 5 illustrates a case where the semiconductor device 110 further includes an electrostatic protection device ESDB. The semiconductor device 110 has a line SLB to which a signal or power is input. The electrostatic protection device ESDB is connected between the line SLB and the ground line GND.

According to the present embodiment, even when the static electricity flows from the conductive member 70 to the lines SLA and SLB other than the ground line GND, the static electricity flows to the ground line GND via the electrostatic protection devices ESDA and ESDB. As a result, it is possible to decrease a damage caused by the static electricity to the laminated body 10 to more securely suppress breakage of the imaging module 3.

Hereinafter, details of FIG. 5 will be described. The signal or power to be input to the lines SLA and SLB corresponds to, for example, the image signal, the driving signal, or the second power signal illustrated in FIG. 14. Although FIG. 5 illustrates merely two electrostatic protection devices, in the example of FIG. 14, three electrostatic protection devices may be provided in correspondence with the image signal, the driving signal, and the second power signal.

The electrostatic protection devices ESDA and ESDB, the ground line GND, and the lines SLA and SLB illustrated in FIG. 5 are formed on the first main plane or the second main plane of the semiconductor device 110 through a semiconductor process. It is sufficient that the ground line GND is connected to the ground line of the cable section 50 via the terminal TGND shown in FIG. 4, for example. That is, the ground line GND is not necessarily the same line with the ground line SGND of FIG. 5.

The electrostatic protection devices ESDA and ESDB are devices that release the static electricity that has entered the lines SLA and SLB to the ground line GND. As illustrated in FIG. 5, the electrostatic protection devices ESDA and ESDB are bidirectional zener diodes, for example. In the electrostatic protection device ESDA, one terminal of the bidirectional zener diode is connected to the line SLA and the other terminal is connected to the ground line GND.

### 4. Second Embodiment

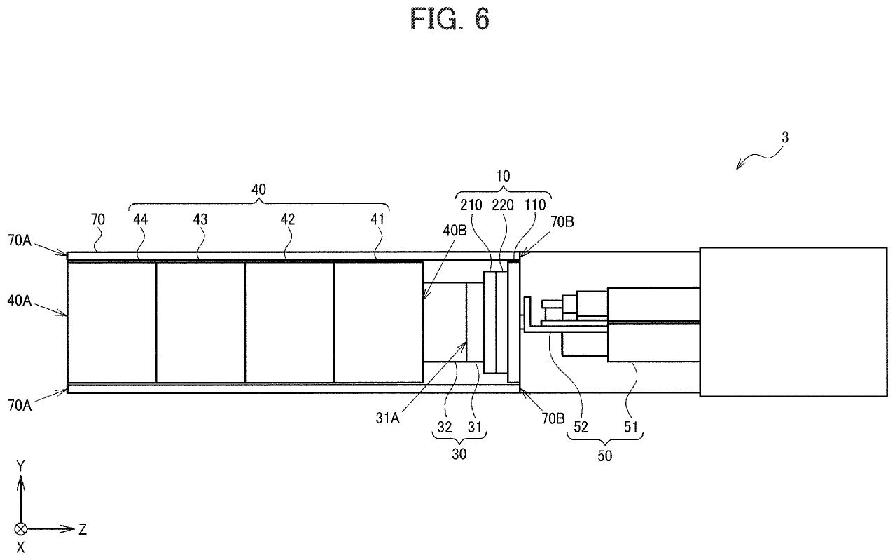

FIG. 6 is a cross-sectional view illustrating a configuration of the imaging module 3 according to a second embodiment. Already-described components are denoted with the same reference numerals, and description of such components is appropriately omitted.

In the laminated body 10, semiconductor devices as a plurality of layers including the semiconductor device 110 as a first layer are laminated. The semiconductor device 110 as the first layer is disposed farther apart from the imager 31 than the semiconductor devices 210 and 220 other than the first layer. In the second embodiment, a distance from an end portion of the semiconductor device 110 as the first layer to an inner wall of the conductive member 70 is equal to or shorter than a distance from end portions of the semiconductor devices 210 and 220 other than the first layer to the inner wall of the conductive member 70. The definition of distance is same as that described in the first embodiment.

According to the present embodiment, of the laminated body 10, the semiconductor device 110 furthest from the imager 31 is closer to the inner wall of the conductive member 70 than the remaining semiconductor devices 210 and 220. As a result, the static electricity that has entered the conductive member 70 flows to the semiconductor device 110 farthest from the imager 31. Accordingly, it is possible to further decrease a risk that the static electricity damages the imager 31 and to more securely suppress breakage of the imager 31.

### 5. Third Embodiment

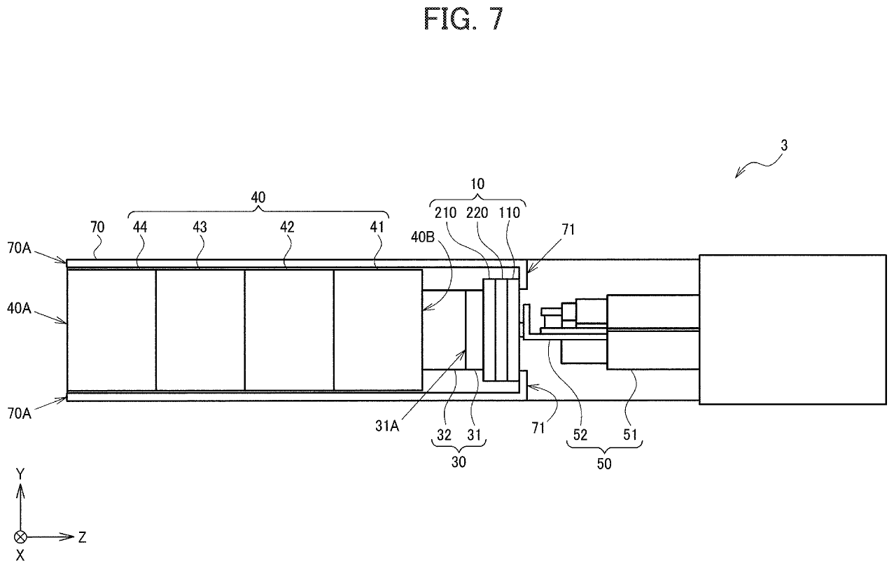

FIG. 7 is a cross-sectional view illustrating a configuration of the imaging module 3 according to a third embodiment. Already-described components are denoted with the same reference numerals, and description of such components is appropriately omitted.

The conductive member 70 has a cylindrical part extending along the optical axis of the objective optical system 40, and a bottom plate 71 provided at one end of the cylindrical part. The bottom plate 71 is made to abut against the second main plane of the semiconductor device 110.

According to the present embodiment, positioning of the conductive member 70 is facilitated in assembling the imaging module 3. In other words, it is sufficient that the laminated body 10, the imager 31, and the objective optical system 40 are inserted from the entering-side end portion 70A of the conductive member 70 and are continued being inserted until the second main plane of the semiconductor device 110 abuts against the bottom plate 71 of the conductive member 70. As a result, a positional relationship between the conductive member 70 and the laminated body 10 or the like is automatically defined. Furthermore, because the bottom plate 71 of the conductive member 71 contacts the second main plane of the semiconductor device 110, the static electricity flows from the conductive member 70 to the semiconductor device 110, which further ensures protection of the imager 31.

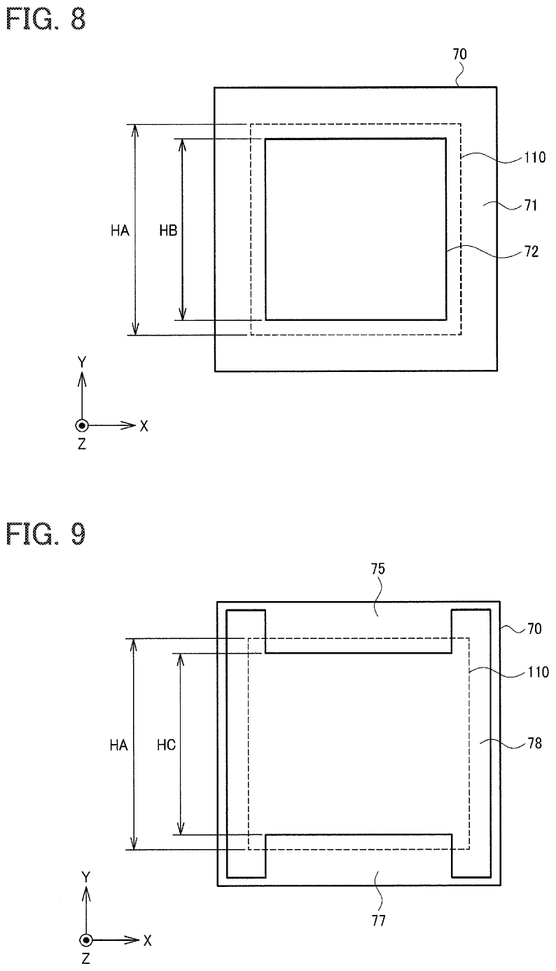

FIG. 8 illustrates the bottom plate 71 and the semiconductor device 110 viewed in the Z direction. In the bottom plate 71, an opening part 72 is provided.

According to the present embodiment, provision of the opening part 72 in the bottom plate 71 facilitates assembly of the imaging module 3. For example, the opening part 72 is larger than the cross section of the cable section 50, and the state of FIG. 7 can be implemented by inserting the cable section 50 from the entering-side end portion 70A of the conductive member 70 into the opening part 72 with the cable section 50 being connected in advance to the second main plane of the semiconductor device 110.

Hereinafter, details of FIG. 7 and FIG. 8 are described. The cylindrical part of the conductive member 70 is a part excluding the bottom plate 71 of the conductive member 70, and same as the conductive member 70 having a cylindrical shape described referring to FIG. 1. The bottom plate 71 is a plate-like member provided in the emitting-side end portion of the cylindrical part and protrudes from the emitting-side end portion of the cylindrical part toward the center of the cylinder. The cylindrical part and the bottom plate 71 may be formed integrally or may be obtained by jointing separate members. According to the third embodiment, the distance between the conductive member 70 and the semiconductor device 110 corresponds to the distance between the bottom plate 71 of the conductive member 70 and the second main plane of the semiconductor device 110, and the distance in the Z direction. Note that the distance between the bottom plate 71 of the conductive member 70 and the second main plane of the semiconductor device 110 may be other than zero.

In FIG. 8, the outer circumference of the opening part 72 is positioned inside the outer circumference of the semiconductor device 110, and a marginal edge part of the semiconductor device 110 entirely abuts against the bottom plate 71. The opening part 72 is square, for example, and a length HB of its side is shorter than a length HA of a side of the semiconductor device 110. When the ground line SGND of FIG. 4 is combined with the third embodiment, the ground line SGND is disposed at a portion abutting against the bottom plate 71, for example, of the second main plane of the semiconductor device 110. Furthermore, it is more preferable that the ground line SGND is disposed at a portion abutting against the bottom plate 71 of the semiconductor device 110 and the bottom plate 71 and the ground line SGND are jointed to each other. Furthermore, it is preferable that the ground line of the cable section 50 is connected to a plane opposite to the semiconductor device 110 of the bottom plate 71.

Note that as illustrated in FIG. 9, a configuration may be adopted in which the marginal edge part of the semiconductor device 110 partially abuts against the bottom plate. FIG. 9 illustrates an example where opposite two sides of four sides of the semiconductor device 110 abut against bottom plates 75 and 77. An opening part 78 has a shape different from that of the semiconductor device 110. The bottom plates 75 and 77 are plate-like members protruding from the side along the X direction of the emitting-side end portion of the cylindrical part toward the center of the cylinder. A distance HC from a boundary between the bottom plate 75 and the opening part 78 to a boundary between the bottom plate 77 and the opening part 78 is shorter than the length HA of the side of the semiconductor device 110.

### 6. Fourth Embodiment

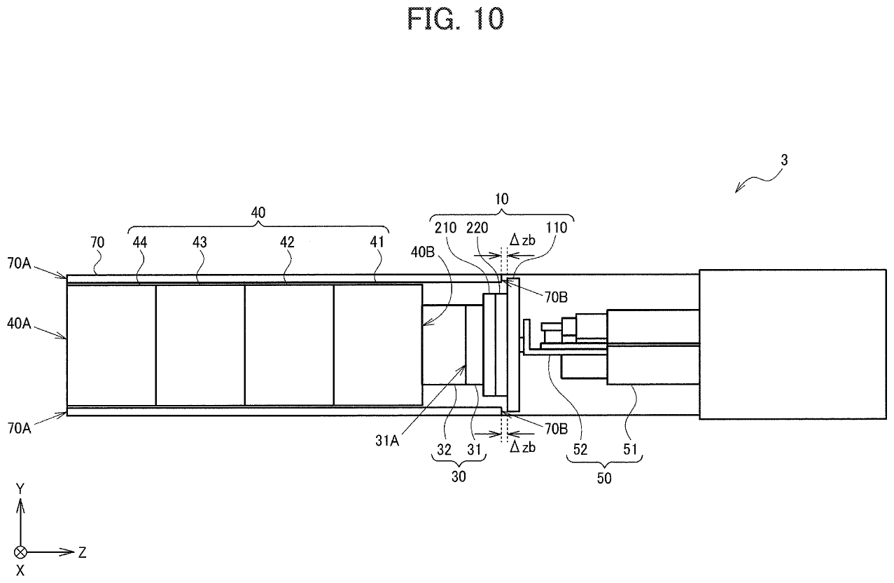

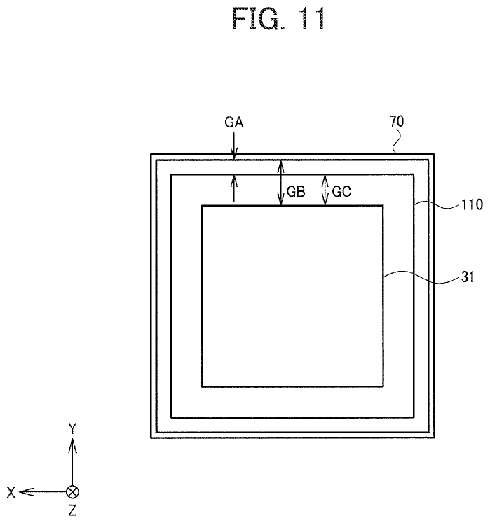

FIG. 10 is a cross-sectional view illustrating a configuration of the imaging module 3 according to a fourth embodiment. Already-described components are denoted with the same reference numerals, and description of such components is appropriately omitted.

According to the fourth embodiment, one end of the conductive member 70 is made to abut against the first main plane of the semiconductor device 110. Specifically, the emitting-side end portion 70B of the conductive member 70 is made to abut against the first main plane of the semiconductor device 110.

The semiconductor device 110 has such a shape viewed in the Z direction that the outer circumference of the semiconductor device 110 exists between the inner circumference and the outer circumference of a wall plane of the conductive member 70. As a result, the emitting-side end portion 70B of the conductive member 70 contacts the first main plane of the semiconductor device 110. FIG. 10 illustrates a case where a distance Δzb between the emitting-side end portion 70B of the conductive member 70 and the first main plane of the semiconductor device 110 is not zero, but Δzb may be zero when the emitting-side end portion 70B and the first main plane completely abut against each other.

According to the present embodiment, positioning of the conductive member 70 is facilitated in assembling the imaging module 3. In other words, it is sufficient that the laminated body 10, the imager 31, and the objective optical system 40 are inserted from the emitting-side end portion 70B of the conductive member 70 and continued being inserted until the first main plane of the semiconductor device 110 abuts against the emitting-side end portion 70B. As a result, a positional relationship between the conductive member 70 and the laminated body 10 and the like is automatically defined. Furthermore, because the emitting-side end portion 70B of the conductive member 70 contacts the first main plane of the semiconductor device 110, the static electricity flows from the conductive member 70 to the semiconductor device 110, which further ensures protection of the imager 31.

### 7. Fifth Embodiment

According to a fifth embodiment, the lateral cross-sectional view of the imaging module 3 is same as that of the first embodiment illustrated in FIG. 1 or the second embodiment illustrated in FIG. 6. FIG. 11 is the imager 31, the semiconductor device 110, and the conductive member 70 viewed in the −Z direction.

As illustrated in FIG. 11, in the direction orthogonal to the optical axis of the objective optical system 40, a distance GA from the end portion of the semiconductor device 110 to the inner wall of the conductive member 70 is shorter than a distance GC from the end portion of the imager 31 to the end portion of the semiconductor device 110. The direction orthogonal to the optical axis of the objective optical system 40 is a direction parallel to the XY plane, which corresponds to the Y direction in FIG. 11. Assuming that a distance from the end portion of the imager 31 to the inner wall of the conductive member 70 is GB, the relation of GB=GA+GC and the relation of GA<GC are satisfied.

According to the present embodiment, the distance GA from the end portion of the semiconductor device 110 to the inner wall of the conductive member 70 can be sufficiently made shorter than the distance GB from the end portion of the imager 31 to the inner wall of the conductive member 70. As a result, it is possible to sufficiently decrease the risk that the static electricity that has entered the conductive member 70 is transferred to the imager 31. Accordingly, protection of the imager 31 can be further ensured.

### 8. Sixth Embodiment

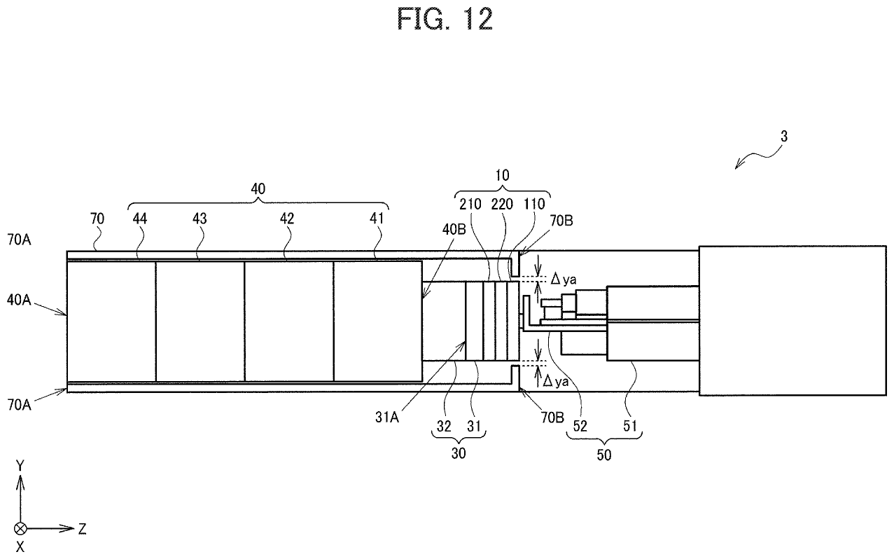

FIG. 12 is a cross-sectional view illustrating a configuration of the imaging module 3 according to a sixth embodiment. Already-described components are denoted with the same reference numerals, and description of such components is appropriately omitted.

According to the sixth embodiment, an inner diameter of a portion adjacent to the semiconductor device 110 in the conductive member 70 is smaller than an inner diameter of a portion adjacent to the imager 31 in the conductive member 70.

Such configuration can also satisfy the following condition: the distance from the end portion of the laminated body 10 to the inner wall of the conductive member 70 is shorter than the distance from the end portion of the imager 31 to the inner wall of the conductive member 70.

Hereinafter, details of FIG. 12 will be described. The portion adjacent to the semiconductor device 110 in the conductive member 70 is a portion adjacent to the semiconductor device 110 in a plane that is parallel to the XY plane and passes through the semiconductor device 110. The inner diameter is a distance, in a plane parallel to the XY plane, from a center of a region surrounded by the conductive member 70 in a cross section parallel to the XY plane to the inner wall, of the conductive member 70, closest to the center. Furthermore, it also can be said that the inner diameter is a radius or a diameter of a circle circumscribing the inner wall of the conductive member 70 in the plane parallel to the XY plane.

In FIG. 12, a thickness of the wall in the emitting-side end portion 70B of the conductive member 70 is larger than a thickness of the wall in another portion. A distance Δya from the end portion of the semiconductor device 110 to the emitting-side end portion 70B of the conductive member 70 is shorter than the distance from the end portion of the imager 31 to the inner wall of the conductive member 70. Note that the portion where the thickness of the wall of the conductive member 70 increases is not limited to the emitting-side end portion 70B. In other words, it is sufficient that the thickness of the wall of a portion adjacent to the entire or partial laminated body 10 in the conductive member 70 is larger compared with another portion. Furthermore, such shape may be adopted that the thickness of the wall of the conductive member 70 is uniform and the wall is narrowed down toward the center of the cylinder at a portion adjacent to the entire or partial laminated body 10.

### 9. Detailed Configuration Example of Sensor Section and Laminated Body

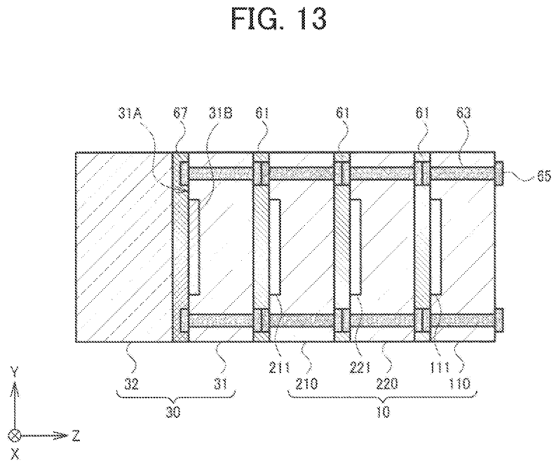

FIG. 13 is a detailed configuration example of the sensor section 30 and the laminated body 10. Hereinafter, description is made to a case where the semiconductor device 110 is provided with an active device and the semiconductor device 210 and the semiconductor device 220 are provided with a passive device. Note that arrangement of the active device and the passive device is not limited to the above-mentioned arrangement, and the active device and the passive device may be provided to any one of the semiconductor devices of the laminated body 10.

To the light receiving plane 31A of the imager 31, the cover glass 32 is adhered using a transparent adhesive 67. As illustrated in FIG. 1, the objective optical system 40 is provided on a side toward the −Z direction relative to the sensor section 30, and a subject image via the objective optical system 40 is formed in the light receiving plane 31A. In the light receiving plane 31A, a light receiving plane 31B is arranged. The light receiving plane 31B is a circuit in which pixel circuits are arranged like a two-dimensional array, and acquires a subject image as a two-dimensional image by photoelectric conversion. The imager 31, the semiconductor device 210, the semiconductor device 220, and the semiconductor device 110 are arranged in this order side by side in the Z direction.

The semiconductor device 110, the semiconductor device 220, and the semiconductor device 210 are respectively laminated through intermediation of sealing resin layers 61. In each of the semiconductor device 110, the semiconductor device 220, and the semiconductor device 210, a through-silicon via (TSV) 63 is formed. Through the TSV 63, the imager 31 and the semiconductor device 210 are connected, the semiconductor device 210 and the semiconductor device 220 are connected, and the semiconductor device 220 and the semiconductor device 110 are connected. The semiconductor device 110 is provided with a first active device 111. The semiconductor device 210 is provided with a first passive device 211. The semiconductor device 220 is provided with a second passive device 221. Note that, although FIG. 13 illustrates an example where the active device or the passive device is provided in the first main plane of the semiconductor device, the active device or the passive device may be provided to the second main plane of the semiconductor device or may be provided to both of the first main plane and the second main plane.

FIG. 14 is a block diagram of the imager 31 and the laminated body 10. The imager 31 includes the light receiving section 31B, a reading section 31C, and a timing generation section 31D. The semiconductor device 210 includes a capacitor C3 as the first passive device 211. The semiconductor device 220 includes a capacitor C4 as the second passive device 211. The semiconductor device 110 includes a signal processing circuit 111A having the first active device 111, and a driving circuit 111B.

The semiconductor device 110 receives, via the cable section 50, input of the power signal, the second power signal, the ground signal, and the driving signal. The driving circuit 111B receives the driving signal and performs processing such as buffering, and then outputs the processed driving signal to the timing generation section 31D. The driving signal is transmitted via the TSVs 63 provided in each layer of the laminated body 10 to the imager 31.

In the semiconductor device 110, the semiconductor device 220, the semiconductor device 210, and the imager 31, a power line L1 for supplying a power signal for the imager 31 is provided. Furthermore, at least the semiconductor device 110 and the semiconductor device 220 are provided with a second power line L3 for supplying the second power signal as a power signal for the signal processing circuit 111A and the driving circuit 111B. Furthermore, the semiconductor device 110, the semiconductor device 220, the semiconductor device 210, and the imager 31 are provided with a ground line L2. The capacitor C3 is provided between the power line L1 and the ground line L2. The capacitor C4 is provided between the second power line L3 and the ground line L2. The capacitors C3 and C4 are by-pass condensers for stabilizing power.

The timing generation section 31D controls operation timing of the light receiving section 31B and the reading section 31C on the basis of the driving signal received from the driving circuit 111B. For example, the driving signal includes a vertical synchronization signal and a horizontal synchronization signal, and the timing generation section 31D generates and outputs a timing pulse signal on the basis of the driving signal. The reading section 31C reads a pixel signal from the pixel circuit, and outputs the read pixel signal to the signal processing circuit 111A. Hereinafter, aggregation of the pixel signals is represented as an image signal. Note that the imager 31 is, for example, a CCD imager or a CMOS imager. The signal processing circuit 111A performs processing to the image signal transmitted from the reading section 31C. The signal processing circuit 111A performs, for example, a noise reduction process, and an A/D conversion process to the image signal. The signal processing circuit 111A outputs the processed image signal to the cable section 50.

According to the embodiments described above, the static electricity that has entered the imaging module 3 flows to the ground line L2 of the laminated body 10 or flows from a signal line such as the image signal or the like via the electrostatic protection device of FIG. 5 to the ground line L2, and then flows from the ground line L2 to the cable section 50. As a result, flowing of the static electricity to the imager 31 can be suppressed, and therefore electrostatic breakage of the light receiving section 31B, the reading section 31C, and the timing generation section 31D of the imager 31 can be suppressed.

### 10. Endoscope and Endoscope System

The imaging module 3 as described above can be incorporated into an endoscope. FIG. 15 is a configuration example of the endoscope system 2 including the endoscope 1 incorporating the imaging module 3.

The endoscope system 2 is provided with the endoscope 1 of the present embodiment, a processor 75A, and a monitor 75B. The endoscope 1 inserts a long and thin insertion section 73 into a body cavity of an inspection subject, and thereby captures an internal body image of the inspection subject and outputs the image signal.

The endoscope 1 is provided with the insertion section 73, a gripping section 74 provided on the side of a proximal end section of the insertion section 73, a universal cord 74B extended from the gripping section 74, and a connector 74C provided on the side of a proximal end section of the universal cord 74B. The insertion section 73 includes a rigid tip section 73A where the imaging module 3 is provided, a bendable curving section 73B provided on the proximal end side of the tip section 73A for changing the direction of the tip section 73A, and a flexible portion 73C extended on the proximal end side of the curving section 73B. The endoscope 1 is a flexible scope, but may be a rigid scope. In other words, the flexible section or the like is not an essential component. The gripping section 74 is provided with a rotatable angle knob 74A serving as an operation section for allowing an operator to operate the curving section 73B.

The universal cord 74B is connected to the processor 75A via the connector 74C. The processor 75A controls the entire endoscope system 2 and performs signal processing to the image signal output by the imaging module 3, and outputs a result of the processing. The monitor 75B displays the image signal output from the processor 75A as an endoscope image.

The tip section 73A of the endoscope 1 has a casing storing the above-mentioned imaging module 3 therein. The casing has a cylindrical shape in which a cross section in the direction crossing the optical axis is circular. For example, inside of the casing made of metal such as stainless steel as a rigid material is filled with a sealing resin such as a silicone resin and an epoxy resin. Note that the external plane of the casing may be covered with a resin layer or the like. Furthermore, the corner of the tip section 73A is chamfered to form a curved line. The casing is preferably made of a light blocking material.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways.

For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope comprising:
an objective optical system;
an imager having a light receiving plane that faces an emitting plane of the objective optical system;
a semiconductor device provided so as to face a plane, of the imager, opposite to the light receiving plane; and
a conductive member that covers the objective optical system, the imager, and the semiconductor device, the conductive member having an external dimension that is identical between a side of the objective optical system and a side of the imager, wherein
a distance from an end portion of the semiconductor device in a direction orthogonal to an optical axis of the objective optical system to an inner wall of the conductive member is shorter than a distance from an end portion of the imager in the direction orthogonal to the optical axis of the objective optical system to the inner wall of the conductive member.

2. The endoscope as defined in claim 1, wherein
a first inner diameter of a portion adjacent to the semiconductor device in the conductive member is identical to a second inner diameter of a portion adjacent to the imager in the conductive member or smaller than the second inner diameter.

3. The endoscope as defined in claim 1, wherein
a first thickness of a portion adjacent to the semiconductor device in the conductive member is identical to a second thickness of a portion adjacent to the imager in the conductive member or larger than the second thickness.

4. The endoscope as defined in claim 1, comprising
a laminated body in which a plurality of semiconductor devices as a plurality of layers are laminated, the plurality of layers including the semiconductor device as a first layer, wherein
the semiconductor device as the first layer among the plurality of layers is disposed further apart from the imager compared with the semiconductor devices other than the first layer among the semiconductor devices as the plurality of layers, and
a distance from an end portion of the semiconductor device as the first layer to an inner wall of the conductive member is equal to or shorter than distances from end portions of the semiconductor devices other than the first layer to the inner wall of the conductive member.

5. The endoscope as defined in claim 1, wherein
the conductive member includes
a cylindrical part along the optical axis of the objective optical system, and
a bottom plate provided on one end of the cylindrical part,
the semiconductor device includes
a first main plane facing the plane, of the imager, opposite to the light receiving plane, and
a second main plane that is a plane opposite to the first main plane, and
the bottom plate is made to abut against the second main plane.

6. The endoscope as defined in claim 5, wherein
the bottom plate is provided with an opening part.

7. An imaging module comprising:
an objective optical system;
an imager having a light receiving plane that faces an emitting plane of the objective optical system;
a semiconductor device provided so as to face a plane, of the imager, opposite to the light receiving plane; and
a conductive member that covers the objective optical system, the imager, and the semiconductor device, the conductive member having an external dimension that is identical between a side of the objective optical system and a side of the imager, wherein
a distance from an end portion of the semiconductor device in a direction orthogonal to an optical axis of the objective optical system to an inner wall of the conductive member is shorter than a distance from an end portion of the imager in the direction orthogonal to the optical axis of the objective optical system to the inner wall of the conductive member.

8. The imaging module as defined in claim 7, wherein
a first inner diameter of a portion adjacent to the semiconductor device in the conductive member is identical to a second inner diameter of a portion adjacent to the imager in the conductive member or smaller than the second inner diameter.

9. The imaging module as defined in claim 7, wherein
a first thickness of a portion adjacent to the semiconductor device in the conductive member is identical to a second thickness of a portion adjacent to the imager in the conductive member or larger than the second thickness.

10. The imaging module as defined in claim 7, comprising
a laminated body in which a plurality of semiconductor devices as a plurality of layers are laminated, the plurality of semiconductor devices including the semiconductor device as a first layer, wherein
the semiconductor device as the first layer among the plurality of layers is
disposed further apart from the imager compared with the semiconductor devices other than the first layer among the semiconductor devices as the plurality of layers, and
a distance from an end portion of the semiconductor device as the first layer to an inner wall of the conductive member is equal to or shorter than distances from end portions of the semiconductor devices other than the first layer to the inner wall of the conductive member.

11. The imaging module as defined in claim 7, wherein
the conductive member includes
a cylindrical part along the optical axis of the objective optical system, and
a bottom plate provided on one end of the cylindrical part,
the semiconductor device includes
a first main plane facing the plane, of the imager, opposite to the light receiving plane, and
a second main plane that is a plane opposite to the first main plane, and
the bottom plate is made to abut against the second main plane.

12. The imaging module as defined in claim 11, wherein
the bottom plate is provided with an opening part.

13. The imaging module as defined in claim 7, wherein
the semiconductor device includes
a first main plane facing the plane, of the imager, opposite to the light receiving plane, and
a second main plane that is a plane opposite to the first main plane, and
one end of the conductive member is made to abut against the first main plane.
